(19) United States
(12) Patent Application Publication
(10) Pub. No.: US 2022/0167886 A1
(43) Pub. Date: Jun. 2, 2022

(54) Chemically Fused Membrane for Analyte Sensing

(71) Applicant: Jeff T. Suri, Fallbrook, CA (US)

(72) Inventor: Jeff T. Suri, Fallbrook, CA (US)

(73) Assignee: Glucovation, Inc., Carlsbad, CA (US)

(21) Appl. No.: 17/537,310

(22) Filed: Nov. 29, 2021

Related U.S. Application Data
(63) Continuation of application No. 17/136,178, filed on Dec. 29, 2020.
(60) Provisional application No. 62/954,793, filed on Dec. 30, 2019.

(51) Int. Cl. A61B 5/145; C12Q 1/00; A61B 5/1486

(52) U.S. Cl. CPC A61B 5/14532; C12Q 1/006; C08F 220/18; A61B 5/14865; C12Q 1/003

(57) ABSTRACT

The invention disclosed herein is a device having an analyte sensor, having a working electrode and a membrane disposed over the electrode and methods of making the device. The multilayered membrane is formed by chemically fusing an inner layer of a polyelectrolyte with an outer layer of an ethylenically unsaturated prepolymer through a chain-growth polymerization reaction of an ethylenically unsaturated silicone prepolymer, a hydride silicone prepolymer, a non-silicone ethylenically unsaturated hydrophilic monomer, a filler and a metal catalyst. The silicone composition formed from the reaction mixture restricts diffusion of an analyte through the membrane. More specifically, the membrane formed comprises a restrictive domain that controls the flux of oxygen and glucose through the membrane to the working electrode.

### CROSS-REFERENCE TO RELATED APPLICATIONS

This application is a continuation-in-part application of patent application Ser. No. 17/136,178 filed 23 Feb. 2021 that claims priority to provisional patent application serial no. 62/954,793 filed Dec. 30, 2019.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### TECHNICAL FIELD

The present invention relates generally to membranes utilized in biological testing and measuring devices. More specifically, the device relates to membranes used with biosensors for the detection and measurement of analytes in biological samples.

### BACKGROUND OF THE INVENTION

Biosensors have become important tools in wearable and self-monitoring devices. Their ability to detect an analyte of interest in vivo and allow for real-time modifications of human behavior and inputs is becoming more important for the development of devices related to personalized medicine and healthcare. Continuous glucose monitoring (CGM) falls into this category and commonly utilizes enzymatic oxidation of glucose with glucose oxidase (GOX) as the chemical detection system (FIG. 1). With commercially available products on the market, CGM has proven to be one of the most valuable approaches to managing diabetes. Finger-stick-based glucometers are becoming a tool of the past as CGM products are beginning to take over the market for effective management of diabetes.

Continuous glucose monitors usually comprise a multilayered membrane with an outer membrane part facing the sample. The outer membrane is a porous or permeable polymer membrane that controls the permeation of analyte and other reactants to the enzyme layer. It may be appreciated that for a first generation glucose sensor (where there is no mediator) the outer membrane of a membrane-biosensor system must allow a sufficient quantity of oxygen molecules into the underlying enzyme layer in order for the sensor output to be proportional to the prevailing glucose concentration in the tissue. For example, at elevated glucose concentrations, a membrane with insufficient oxygen permeability (or too high a glucose permeability) may generate a current that plateaus as the glucose concentration continues to rise above normal concentrations. Often, in persons with diabetes mellitus, glucose concentrations may rise to very high concentrations, even exceeding 400 mg/dL in blood. Thus, in a first generation glucose sensor, the membrane should control the permeation of the reactants glucose and oxygen. However, in the blood or interstitial fluid the glucose concentration exceeds the oxygen concentration by orders of magnitude. Thus, the main objective of the outer membrane is to reduce the permeation of glucose to the enzyme layer. The same applies for measurements of other biomolecules.

There are various requirements needed for polymeric membranes to be effective in vivo. Membranes must be highly permeable to oxygen, robust towards reactive oxygen species (ROS) and degradative enzymes such as proteases and oxidases and resistant towards polymeric rearrangement and surface chemistry changes. All of these requirements are necessary in order avoid signal drift of the device and allow for the accurate measurement of glucose concentrations in vivo without the constant re-calibration of the sensor.

Various groups have addressed these requirements in different ways. U.S. Pat. No. 5,322,063 teaches that various compositions of hydrophilic polyurethanes may be used to control the ratios of the diffusion coefficients of oxygen to glucose in an implantable glucose sensor. U.S. Pat. Nos. 5,428,123, 5,589,563, and 5,756,632 describe the use of materials in an indwelling glucose sensor application and found that the requirement for high oxygen and glucose permeability was at conflict with the requirement for structural strength and integrity. More specifically, it was found that when the material was made sufficiently oxygen permeable, it became too weak and tended to break apart on the sensor after being present in the body's interstitial fluid for more than a few hours. Within biological solutions, such as blood or interstitial fluid, there exist a number of reactive materials and enzymes that may bring about cleavage of the polymer's molecular chains through hydrogen abstraction, addition, and electron transfer reactions resulting in loss of membrane strength and integrity. This loss of membrane overall integrity may be detrimental to applications that depend on the permselectivity of the polymeric material and the exclusion of solids and larger biological molecules, such as the detection of the levels of glucose within the body fluids of a living human body.

Efforts have been made to prepare silicone membrane systems for transcutaneous glucose sensors. Silicones are polymers containing alternating silicon and oxygen atoms in the backbone and having various functional groups attached to the silicon atoms of the backbone. Silicone copolymers include backbone units that possess a variety of groups (e.g., polyether or polyurethane) attached to the silicone atoms. Both silicones and silicone copolymers are useful materials for a wide variety of applications (e.g., rubbers, adhesives, sealing agents, release coatings, antifoam agents). Because of their biocompatibility, silicones present a low risk of unfavorable biological reactions and have therefore gained the medical industry's recognition as being useful in a wide variety of medical devices. However, silicone is an inherently hydrophobic material, and therefore does not permit the transport of glucose and other such water-soluble molecules.

United States patent application serial no. 20050090607 teaches that silicone grafted with PEG onto the main chain provides a hydrophilic glucose permeable membrane. This membrane was prepared by mixing vinylic PEG-substituted silicone prepolymers with silicone hydride prepolymers and curing in the presence of platinum (Pt) at elevated temperature.

U.S. Pat. No. 9,549,693 teaches that silicone blended with PEO/PPO surfactants provides a glucose permeable silicone membrane. However, this membrane is susceptible to polymeric rearrangement that leads to changes in the glucose permeability and ultimately to signal drift.

United States patent application serial no. 20110152654 teaches that a blended mixture of polyurethane and branched silicone acrylate polymer provides a glucose permeable membrane. However, this system is fairly complex and difficult to control because it is created through free radical polymerization and results in the formation of an interpenetrating network. This makes it difficult to generate reproducible glucose limiting membranes.

One of the most common reactions exploited for the preparation of polysiloxanes (silicones) is the hydrosilylation reaction (FIG. 2). Typically referred to as a room temperature volcanization (RTV) or the product of addition-cure, silicone elastomers are prepared by mixing a Part A, vinyl functional silicone, filler, and catalyst with a Part B hydride functional silicone in specific proportions. The mixture can be heated or cured at room temperature depending on the functional groups and catalyst. In this context, various authors have reported that end functional hydride polydimethylsiloxane, (h₂PDMS) and copoly-(dimethyl)(methyl-hydrogen)siloxane can be modified with different functional groups via the hydrosilyation reaction (Putzien, S.; Nuyken, O.; and Kuhn, F. E. Prog. Polym. Sci., 2010, 35, 687-713). The preparation of acrylate containing (Kokko, B. J. J. Appl. Polym. Sci., 1993, 47, -1309-1314) and fluorinated PDMS (Boutevin, B.; Guida-Pietrasanta, F.; and Ratsimihety, A. J. Polym. Sci., Part A: Polym. Chem., 2000, 38, 3722-3728) using this method has also been reported. Hydrosilylation of h₂PDMS with (meth)acrylic acid (Mukbaniani, O.; Zaikov, G.; Pirckheliani, N.; Tatrishvili, T.; Meladze, S.; Pachulia Z.; and Labartkava, M. J. Appl. Polym. Sci., 2007, 103, 3243-3252 and Cheng, L. J.; Liu, Q. Q.; Zhang, A. Q.; Yang L.; and Lin, Y. L.; J. Macromol. Sci., Part A: Pure Appl. Chem., 2014, 51, 16-6), amine, epoxy, and methacrylate terminal end groups (Chakraborty R. and Soucek, M. D. Macromol. Chem. Phys., 2008, 209, 604-614) has also been prepared. In another study, linear telechelic h₂PDMS was modified with methacrylates bearing different end groups (Risangud, N.; Li, Z.; Anastasaki, A.; Wilson, P.; Kempea, K. RSC Adv. 2015, 5, 5879-5885).

Although CGMs have proven to be effective there is still room for technological improvement. Easy-to-use devices still need to be developed that remain accurate over their use-life and are relatively inexpensive.

One factor that impacts the accuracy and ease of use for CGMs is their stability and requirement for calibration. Calibration is necessary because of either (A) changes that take place in the physiologic environment around the biosensor or (B) because of changes in the sensor chemistry itself as a function of time or environment. Although dealing with (A) can be challenging and variable, (B) can be addressed via the proper design of the biosensor chemistry.

Electrochemically-based CGMs usually comprise a multilayered membrane with an outer membrane facing the bodily fluid or tissue. While the outer membrane controls the permeation of analyte, the inner membrane, sometimes called the enzyme layer, functions to react with the analyte to produce a product that further reacts with the surface of the electrode. For this type of sensing system, it is critical that the enzyme is properly immobilized within the inner layer. This will ensure stable signal and longevity of the sensor.

There are various approaches to immobilizing the enzyme in the inner layer. For a first generation glucose sensor (where there is no mediator) the enzyme layer serves to provide a stable environment for the enzyme to function properly without any inhibition of its active site. It also needs to contain a sufficient quantity of oxygen molecules in order for the sensor output to be proportional to the prevailing glucose concentration in the tissue. All of these requirements are necessary in order to avoid signal drift of the device and allow for the accurate measurement of glucose concentrations in vivo without the constant re-calibration of the sensor.

Various groups have addressed these requirements in different ways. U.S. Pat. No. 9,737,250 claims that addition of polyvinylpyrrolidone (PVP) to the GOX layer improves oxygen permeability and stability of the sensor. It is reported that the addition of one or more hydrophilic polymers in the enzyme layer results in improved sensor performance (i.e., less signal drift) under low oxygen conditions.

U.S. Pat. No. 8,280,474 claims that in order to improve the stability and lifetime of a sensor the Ag/AgCl reference electrode is covered with an impermeable dielectric layer or a permselective coating that decreases the solubility of the AgCl to the surrounding aqueous environment, thereby improving the stability and longevity of the electrochemical sensor.

U.S. Pat. No. 7,090,756 describes the use of trifunctional crosslinkers to help stabilize transition metals for a wired enzyme sensor. Inadequate crosslinking of a redox polymer can result in excessive swelling of the redox polymer and to the leaching of the components. The crosslinkers form a tighter network and inhibit leaching of the metals from the sensor. This provides for a more stable sensor signal.

PCT 2017/189764 describes the use of a glucose oxidase bioconjugate that is UV-cured into a polymer matrix to provide more long-term (i.e., over 6 days) stability during in vivo sensor operation.

U.S. Pat. No. 8,608,921 describes the use of multilayered membrane consisting of an electrode layer covered with an analyte sensing layer and an analyte modulating layer that functions in analyte diffusion control. Blends of polyurethane/polyurea and a polymeric acrylate for the analyte sensing layer were found to allow for the ability to eliminate the need for a separate adhesion promoting material disposed between various layers of the sensor (e.g. one disposed between the analyte sensing layer and the analyte modulating layer). This helped overcome hydration challenges and the sensor's ability to provide accurate signals that correspond to the concentrations of glucose.

U.S. patent application serial no. US20140012115A describes the use of adhesion promoting (AP) layers in between an analyte sensing layer and an analyte modulating layer in order to address problems associated with sensor layers delaminating and/or degrading over time in a manner that can limit the functional lifetime of the sensor. The AP layers are formed by plasma treatment and hexadimethylsiloxane treatment of the analyte modulating layer.

U.S. Pat. Nos. 6,514,718, 5,773,270, and 4,418,148 describe how the use of a multilayered membrane gives optimal response stability, good mechanical strength, and high diffusion resistance for unexpected species and macromolecules.

U.S. Pat. No. 7,799,191B2 describes the preparation of an enzyme layer formed on the surface of an electrode that is covered by an epoxy polymer layer. The epoxy polymer layer covers the immobilized enzyme layer in order to add durability to the underlying enzyme layer, while also, in certain configurations, serves as a diffusion barrier to the internal enzyme layer.

Although these different approaches are geared towards stabilizing the enzymatic-based glucose sensor, there is still an unmet need of a stable membrane system that does not change over time. Conventional methods of enzyme immobilization within multilayered membranes still lack long term stability and operability. The current invention addresses this current problem by providing for a chemical fusing of multilayered membranes such that the chemically fused membranes impart a greater stability to the sensor signal and accuracy.

The forgoing examples of related art and limitations related therewith are intended to be illustrative and not exclusive, and they do not imply any limitations on the. invention described and claimed herein. Various limitations of the related art will become apparent to those skilled in the art upon a reading and understanding of the specification below and the accompanying drawings.

### SUMMARY OF THE INVENTION

The device herein disclosed and described provides an analyte sensor comprising a working electrode and a membrane disposed over the electrode. The membrane is formed from a silicone composition reaction mixture of an ethylenically unsaturated silicone prepolymer, a hydride silicone prepolymer, a non-silicone ethylenically unsaturated hydrophilic monomer, a filler and a metal catalyst. The silicone composition formed from the reaction mixture restricts diffusion of an analyte through the membrane. More specifically, the membrane formed comprises a restrictive domain that controls the flux of oxygen and glucose through the membrane and wherein the silicone composition formed has the structure:

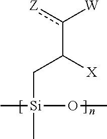

wherein X is H or an alkyl; Z is O, H₂; W is OH, O-alkyl, O-alkylhydroxy, O-alkylalkoxy, O-methacrylate, O-acrylate, and n is >1 or structure:

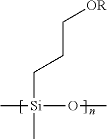

wherein R is H or —(CH₂CH₂O)ₘ—CH₂CH₂OH; n is ≤1; and m is >0.

Another aspect of the present invention is a method of making an analyte sensor, comprising the steps of disposing a sensing layer on a surface, applying a membrane over the sensing layer by coating with a silicone solution and curing the coated silicone solution at a temperature range of between 4° C. to 80° C. The membrane being prepared from a silicone composition reaction mixture of an ethylenically unsaturated silicone prepolymer, a hydride silicone prepolymer, a non-silicone based ethylenically unsaturated hydrophilic monomer a filler and a metal catalyst.

In another embodiment, the method of making the analyte sensor of the present invention may comprise the steps of disposing a first layer on a substrate, wherein the first layer is formed in a crosslinking reaction, chemically modifying the first layer with ethylenically unsaturated groups and then disposing a subsequent layer comprising an ethylenically unsaturated prepolymer, wherein the subsequent layer is formed in a chain-growth polymerization reaction. The polymerization reaction may be a platinum cured hydrosilylation reaction or a free radical reaction, wherein the free radical reaction in initiated by a photo-initiator or a thermal-initiator.

In one embodiment of either aspect of the invention, the ethylenically unsaturated silicone prepolymer comprises about 40 to about 90 percent of the membrane. More specifically, the ethylenically unsaturated silicone prepolymer comprises about 40, about 45, about 50, about 55, about 60, about 65, about 70, about 75, about 80, about 85 or about 90 percent of the membrane formed from the silicone composition reaction mixture.

In another embodiment of either aspect of the invention, the hydride silicone prepolymer comprises about 5 to about 20 percent of the membrane. More specifically, the hydride silicone prepolymer comprises about 5, about 6, about 8, about 10, about 12, about 14, about 16, about 18 or about 20 percent of the membrane formed from the silicone composition reaction mixture.

In another embodiment of either aspect of the invention, the non-silicone ethylenically unsaturated hydrophilic monomer is comprised of functional groups comprising hydroxy, ethoxy, methoxy, ethylene oxide, polypropylene oxide, methacrylate, acrylate and carboxylic acid as well as alkyl and ether main chain groups. More specifically, the monomer is allyl alcohol, 2-allyloxyethanol, 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid and/or acrylic acid.

In another embodiment of either aspect of the invention, W of the silicone composition form may be —OM, —O—(CH₂)ₘ, —O(CH₂)₂—OH, —O—CH₂—(CH₂)₂O, —(O—CH₂—CH₂)ₘ—O—C═O—C(CH₂)(CH₃), wherein n is >1, m is ≥0 and M is Na, K or H.

In another embodiment of either aspect of the invention, the non-silicone ethylenically unsaturated hydrophilic monomer comprises about 2 to about 30 percent of the membrane. More specifically, the non-silicone ethylenically unsaturated hydrophilic monomer comprises about 2, about 4, about 6, about 8, about 10, about 12, about 15, about 20, about 24, about 28 or about 30 percent of the membrane formed from the silicone composition reaction mixture.

In another embodiment of either aspect of the invention, the filler comprises about 2 to about 40 percent of the membrane. More specifically, the filler comprises about 2, about 4, about 8, about 10, about 16, about 20, about 25, about 30, about 35 or about 40 percent of the membrane formed from the silicone composition reaction mixture.

In another aspect of the present invention, an analyte sensor is provided comprising a working electrode and a multilayered membrane disposed over the electrode wherein the membrane is formed from a reaction mixture comprising a first sensing layer of an ethylenically unsaturated polyelectrolyte prepolymer and a subsequent flux limiting layer of an ethylenically unsaturated prepolymer and a hydride prepolymer, wherein the layers are covalently attached to each other.

In another embodiment the analyte sensor above further comprises a reference electrode wherein the reference electrode contains iridium, iridium oxide, rhodium or rhodium oxide.

In yet another embodiment the analyte sensor above further comprises an enzyme, wherein the enzyme is glucose oxidase, glucose dehydrogenase, catalase, 3-hydroxybutyrate dehydrogenase and/or β-hydroxybutyrate dehydrogenase.

In yet another embodiment the polyelectrolyte of the analyte sensor above is a carboxylic acid and wherein the carboxylic acid is a polyacrylic acid or a polyurethane.

In yet another embodiment the sensing layer of the analyte sensor above may be formed through a crosslinking reaction, wherein the crosslinker is an aziridine and the aziridine is trimethylolpropanetris(2-methyl-1-aziridinepropionate), pentaerythritoltris(3-(1-aziridine)propionate or N,N′(methylenedi-p-phenylene)bis(aziridine-1-carboxiamide).

In yet another embodiment, the flux limiting layer of the analyte sensor above comprises an ethylenically unsaturated silicone prepolymer that may be a vinyl functional polysiloxane, ethylenoxide functional polysiloxane or tetrahydrofurfuryloxypropyl siloxane and may further comprise functional groups consisting of hydroxyl, ethoxy, methoxy, ethylene oxide, propylene oxide, methacrylate, acrylate and/or carboxylic acids. The flux limiting layer may comprise 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid, acrylic acid, allyl alcohol, 2-allyloxyethanol, ethylenoxide terminated monovinylpolysiloxane and/or tetrahydrofurfuryloxypropyl terminated monovinylpolysiloxane.

In another aspect of the present invention, the device provides an analyte sensor, having a working electrode and a multilayered membrane disposed over the electrode. The membrane is formed by covalently attaching an outer layer comprised of an ethylenically unsaturated prepolymer to an inner layer comprised of an ethylenically unsaturated polyelectrolyte and an enzyme. The final fused membrane composition acts a sensor membrane that provides a more stable and robust system. More specifically, the multilayered membrane formed comprises a restrictive domain that controls the flux of oxygen and glucose through the membrane to the working electrode without significant drift in sensor signal.

Another aspect of the present invention is a method of making an analyte sensor, comprising the steps of disposing a sensing layer on a surface, treating the sensing layer with a coupling agent and attaching ethylenically unsaturated functional groups, and applying another layer over the sensing layer and curing the coated solution at a temperature range of between 4° C. to 80° C. The membrane being prepared from a composition reaction mixture of a polyelectrolyte mixed with an enzyme and a crosslinker as a first layer that is functionalized with ethylenically unsaturated groups and chemically reacted with an outer layer comprised of an ethylenically unsaturated prepolymer.

In one embodiment of either aspect of the invention, the polyelectrolyte comprises about 1 to about 10 percent of the membrane. More specifically, the polyelectrolyte comprises about 1, about 2, about 3, about 4, about 5, about 6, about 7, about 8, about 9, about 10 percent of the membrane formed from the composition reaction mixture.

In one embodiment of either aspect of the invention, the enzyme comprises about 1 to about 10 percent of the membrane. More specifically, the enzyme comprises about 0.5, about 1, about 2, about 3, about 4, about 5, about 6, about 7, about 8, about 9, about 10 percent of the membrane formed from the composition reaction mixture.

In another embodiment of either aspect of the invention, the crosslinker comprises about 0.1 to about 5 percent of the membrane. More specifically, the crosslinker comprises about 0.1, about 0.2, about 0.3, about 0.4, about 0.5, about 0.6, about 0.7, about 0.8, about 0.9, about 1, about 1.2, about 1.5, about 2 about 2.5, about 3, about 3.5, about 4, about 4.5, about 5 percent of the membrane formed from the crosslinker composition reaction mixture.

In another embodiment of either aspect of the invention, the polyelectrolyte is comprised of carboxylic acid, hydroxy, and amino functional groups.

In another embodiment of either aspect of the invention, the ethylenically unsaturated monomer that is attached to the sensing layer is comprised of hydroxy, methacrylate, acrylate, vinyl, and ester end groups; and alkyl and ether main chain groups. More specifically, the monomer is allyl alcohol, 2-allyloxyethanol, 2-hydroxyethyl methacrylate, 3-hydroxypropyl methacrylate, glycidyl methacrylate, diethyleneglycol monomethacrylate, diethylene glycol methyl ether methacrylate, polyethylene glycol monomethacrylate, polyethylene glycol monoacrylate, allyl methacrylate, methacrylic acid and/or acrylic acid.

In one embodiment of either aspect of the invention, the coupling agent comprises a carbodiimide functionality.

Another aspect of the present invention is an electrochemical sensing system that includes a working electrode and a reference electrode, wherein the working electrode contains a biosensing molecule disposed on its surface and is operative to catalytically decompose a non-electroactive target analyte to yield an electroactive by-product. The biosensing molecule is covered by a membrane formed from a silicone composition reaction mixture of an ethylenically unsaturated silicone prepolymer, a hydride silicone prepolymer, an ethylenically unsaturated hydrophilic monomer, a filler and a metal catalyst. The silicone composition formed from the reaction mixture restricts diffusion of an analyte through the membrane. More specifically, the membrane formed comprises a restrictive domain that controls the flux of oxygen and glucose through the membrane to the working electrode.

The reference electrode provides: a stable reference voltage for the working electrode; is not consumed by the oxidation or reduction reaction at its surface when a metal oxide is utilized, thereby providing longer operational life; has no usage limitations due to reference consumption; and substantially reduces signal drift. Suitable materials for the reference electrode are, for example, metal oxides (e.g., iridium oxide, ruthenium oxide, palladium oxide, platinum oxide, rhodium oxide), conducting polymers (e.g., polyethylene dioxythiophene:polystyrene sulfonate (PEDOT:PSS), and any other suitable stable reference electrode or combination thereof. In some embodiments, the reference electrode can include rhodium or rhodium oxide, iridium or iridium oxide.

In other embodiments, the membrane is configured to reduce flux of an analyte or at least one interferent to the sensing layer, wherein a biocompatible layer is disposed over the multilayer membrane and wherein the analyte sensor is adapted for implantation of at least a portion of the sensor into an animal, wherein the implantation is subcutaneous.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

The objects, features, and advantages of the invention will be brought out in the following part of the specification, wherein detailed description is for the purpose of fully disclosing the invention without placing limitations thereon.

### BRIEF DESCRIPTION OF THE DRAWINGS

FIG. 1 shows the enzymatic oxidation of glucose with glucose oxidase.

FIG. 2 shows the hydrosilylation mechanism catalyzed by a transition metal.

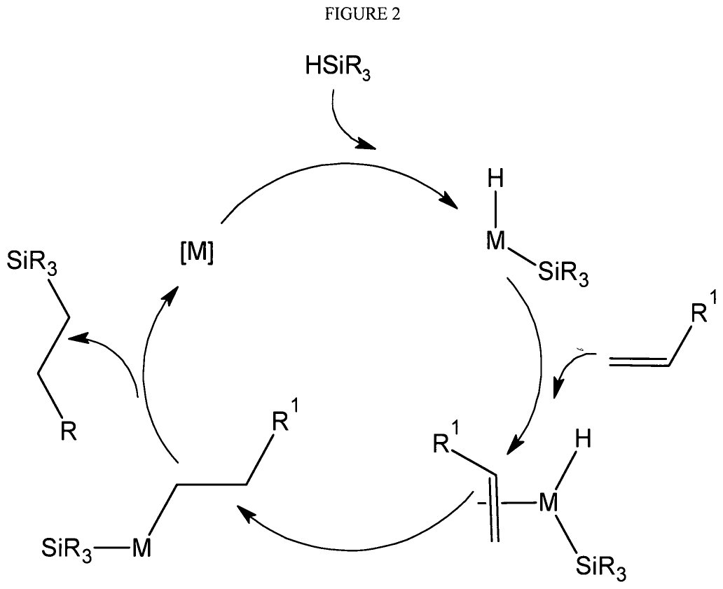

FIG. 3 shows the hydrosilyation reaction between vinylsilicone prepolymers and ethylenically unsaturated monomers.

FIG. 4 shows non-silicone ethylenically unsaturated monomers and their silicone products.

FIG. 5 shows the amperometric glucose response of a sensor wire coated with crosslinked glucose oxidase and different membrane materials.

FIG. 6 shows the glucose response curve.

FIG. 7 shows the reaction of enzyme with crosslinker aziridine and polyacrylic acid.

FIG. 8 shows the EDC coupling reaction of 2-hydroxyethylmethacrylate to polyacrylic acid-enzyme polymer to create an ethylenically unsaturated enzyme prepolymer composition.

FIGS. 9 A and B shows the concept of covalently attaching separate membrane layers via polymerization of their ethylenically unsaturated monomers.

FIG. 10 shows a comparison of glucose response curves for sensor wires treated with EDC/HEMA and not treated with EDC/HEMA.

FIG. 11 shows the percentage change in sensor sensitivity of a series of sensor wires treated with EDC/HEMA in comparison to no EDC/HEMA treatment.

### DETAILED DESCRIPTION OF THE INVENTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

As used herein, the term “alkyl” refers to a single bond chain of hydrocarbons ranging, in some embodiments, from 1-20 carbon atoms, and ranging in some embodiments, from 1-8 carbon atoms; examples include methyl, ethyl, propyl, isopropyl, n-butyl, sec-butyl, isobutyl, tert-butyl, pentyl, isoamyl, hexyl, octyl, dodecanyl, and the like.

The term “analyte” as used herein, refers to a substance or chemical constituent in a biological fluid (e.g., blood, interstitial fluid, cerebral spinal fluid, lymph fluid or urine) that can be analyzed. Analytes include naturally occurring substances, artificial substances, metabolites, and/or reaction products. In some embodiments, the analyte for measurement by the sensor is glucose.

The terms “sensor” or “sensing” as used herein is a description of the component or region of a device by which an analyte can be quantified.

The term “domain” as used herein, describes regions of the membrane that may be layers, uniform or non-uniform gradients (e.g., anisotropic), functional aspects of a material, or provided as portions of the membrane.

The term “hydrophilic,” as used herein, describes a material, or portion thereof, that will more readily associate with water than with lipids. Representative hydrophilic groups include but are not limited to hydroxy, ethylene oxide, propylene oxide, amino, amido, imido, carboxyl, sulfonate, ethoxy, and methoxy.

The term “silicone” as used herein, describes a composition of matter that comprises polymers having alternating silicon and oxygen atoms in the backbone. Examples include, but are not limited to, vinyl terminated polydimethylsiloxane and vinylmethylsiloxane copolymer.

The term “prepolymer”, (e.g., “polyelectrolyte prepolymer” or “ethylenically unsaturated silicone prepolymer”) as used herein, describes a composition of matter and refers to a monomer or system of monomers that have been reacted to an intermediate molecular mass state. This material is capable of further polymerization by reactive groups to a fully cured high molecular weight state. Examples include but are not limited to vinyl terminated polydimethylsiloxane and vinylmethylsiloxane copolymer, polyacrylic acid, vinylsiloxane, and polyethyleneglycol dimethacrylate.

The phrase “ethylenically unsaturated” as used herein, describes a composition of matter that comprises a carbon-carbon double bond that can be further reacted. Examples include but are not limited to 2-hydroxyethyl methacrylate and polyethyleneglycol dimethacrylate.

The phrase “hydride silicone” as used herein, describes a composition of matter that comprises a siloxane polymer with at least one Si—H functional group. Examples include, but are not limited to, methylhydrosiloxane-dimethylsiloxane copolymer, trimethylsiloxane terminated and hydride terminated polydimethylsiloxane.

The term “HEMA” as used herein, refers to 2-hydroxyethyl methacrylate.

The term “aziridine” as used herein, refers to compounds containing one or more of the aziridine functional group; a three-membered heterocycle with one amine (—NR—) and two methylene bridges (—CR₂—). Examples include but are not limited to N,N′-(methylenedi-p-phenylene)bis(aziridine-1-carboxamide) and trimethylolpropane tris(2-methyl-1-aziridine propionate).

The term “crosslinker” as used herein, refers to compounds used to connect two or more polymer chains. Examples included but are not limited to aziridines, epoxides, aldehydes, and carbodiimides.

The term “filler” as used herein, describes a type of material that provides reinforcement for a polymeric membrane. Examples include but are not limited to fumed silica, precipitated or wet silica, ground quartz, aluminum hydroxides (aluminum trihydrate), carbon black, diatomaceous earth, clay, and Kaolin.

The term “coupling agent” as used herein, refers to compounds that connect molecules to each other via a coupling reaction. Examples include but are not limited to 1-ethyl-3-(-3-dimethylaminopropyl) carbodiimide hydrochloride; N′, N′ -dicyclohexyl carbodiimide; 1,1′-Carbonyldiimidazole; and (1-[Bis(dimethylamino)methylene]-1H-1,2,3-triazolo [4,5-b]pyridinium 3-oxide hexafluorophosphate.

The invention disclosed herein provides a glucose sensor membrane that solves the problems of the previous membranes both in terms of potential in vivo problems and in terms of membrane preparation in that it restricts glucose diffusion, is highly oxygen permeable, is mechanically strong, forms a crosslinked polymer network, is highly biocompatible, is stable over time, and may be prepared as a dip-coating.

In one aspect of the present invention, the device herein disclosed and described provides an analyte sensor, comprising: a working electrode and a membrane disposed over the electrode. The membrane is formed from a silicone composition reaction mixture of an ethylenically unsaturated silicone prepolymer, a hydride silicone prepolymer, a non-silicone ethylenically unsaturated hydrophilic monomer, a filler, and a metal catalyst. The silicone composition formed from the reaction mixture restricts diffusion of an analyte through the membrane. More specifically, the membrane formed comprises a restrictive domain that controls the flux of oxygen and glucose through the membrane to the working electrode.

Another aspect of the present invention, is a method of making an analyte sensor, comprising the steps of disposing a sensing layer on a surface, applying a membrane over the sensing layer by coating with a silicone solution and curing the coated silicone solution at a temperature range of between 4° C.-80° C. The membrane being prepared from a silicone composition reaction mixture of an ethylenically unsaturated silicone prepolymer, a hydride silicone prepolymer, a non-silicone ethylenically unsaturated hydrophilic monomer, a filler; and a metal catalyst.

Embodiments of the invention include a sensor having a plurality of layered elements including an analyte limiting membrane comprising a transition metal cured crosslinked silicone. Such polymeric membranes are particularly useful in the construction of electrochemical sensors for in vivo use, and embodiments of the invention include specific biosensor configurations that incorporate these polymeric membranes. The membrane embodiments of the invention allow for a combination of desirable properties including: permeability to molecules such as glucose over a range of temperatures, good mechanical properties of use as an outer polymeric membrane, and good processing properties for in situ preparation on a substrate. Consequently, glucose sensors that incorporate such polymeric membranes show an enhanced in vivo performance profile.

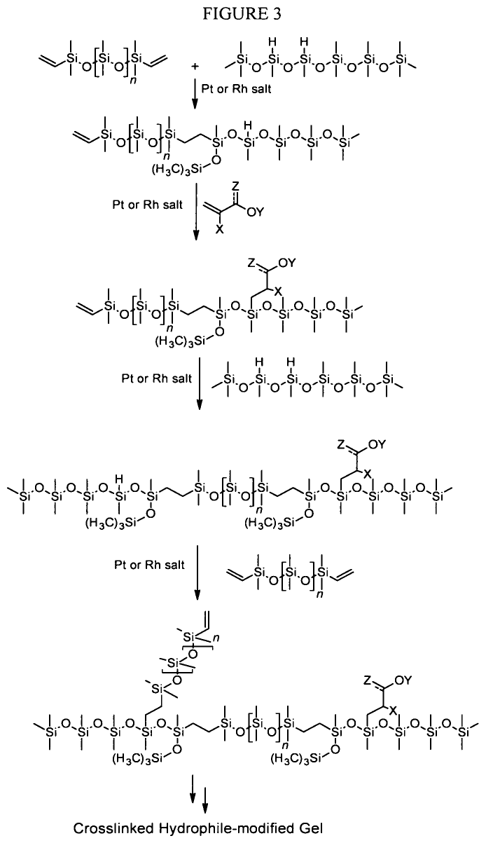

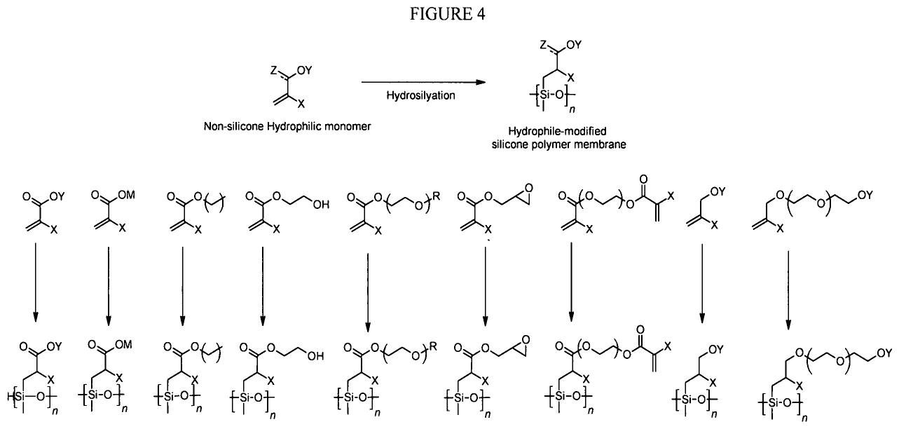

In some embodiments of the present invention the hydrophile-modified silicone may comprise the following group:

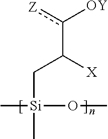

wherein n is >1, X is H, alkyl; Z is O, H₂; and Y is H, alkyl, alkylhydroxy, alkylalkoxy, acrylate, methacrylate. Depending on the non-silicone hydrophilic monomer selected for the hydrosilylation reaction, a number of hydrophile-modified silicones may be produced. Some of these are shown in FIGS. 3 and 4.

The ethylenically unsaturated silicone prepolymer may comprise about 40 to about 90 percent of the membrane. More specifically, the ethylenically unsaturated silicone prepolymer may comprise about 40, about 45, about 50, about 55, about 60, about 65, about 70, about 75, about 80, about 85 or about 90 percent of the membrane formed from the silicone composition reaction mixture.

The hydride silicone prepolymer may comprise about 5 to about 20 percent of the membrane. More specifically, the hydride silicone prepolymer may comprise about 5, about 6, about 8, about 10, about 12, about 14, about 16, about 18 or about 20 percent of the membrane formed from the silicone composition reaction mixture.

The non-silicone ethylenically unsaturated hydrophilic monomer may be comprised of hydroxy, alkoxy, epoxy, vinyl, and carboxylic acid end groups; and alkyl and ether main chain groups. More specifically, the monomer may be allyl alcohol, 2-allyloxyethanol, 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid, acrylic acid. Further, the non-silicone ethylenically unsaturated hydrophilic monomer may comprise about 2 to about 30 percent of the membrane. More specifically, the non-silicone ethylenically unsaturated hydrophilic monomer may comprise about 2, about 4, about 6, about 8, about 10, about 12, about 15, about 20, about 24, about 28 or about 30 percent of the membrane formed from the silicone composition reaction mixture.

The filler may comprise about 2 to about 40 percent of the membrane. More specifically, the filler may comprise about 2, about 4, about 8, about 10, about 16, about 20, about 25, about 30, about 35 or about 40 percent of the membrane formed from the silicone composition reaction mixture.

The continuous glucose monitoring system described herein is inserted underneath the skin with a small needle. The needle is removed and the sensor resides in the interstitial fluid and comes in direct contact with fluid containing glucose. The glucose permeates through the sensor membrane and reacts with glucose oxidase generating hydrogen peroxide that is then detected amperometrically. Similar systems are described in In Vivo Glucose Sensing, Cunningham, D. D., Stenken, J. A., Eds; John Wiley & Sons, Hoboken, N.J., 2010.

The unexpected result is that when a methacrylate (i.e., non-silicone based hydrophilic) monomer is mixed with a silicone hydride prepolymer, a vinyl silicone prepolymer, and a metal (e.g., platinum or rhodium) catalyst, a silicone membrane is formed in situ that is glucose and oxygen permeable, biocompatible, and robust towards processing steps required to build an electrochemical sensor.

Another aspect of the present invention herein disclosed and described is an analyte sensor, having a working electrode and a multilayered membrane disposed over the electrode. The membrane is formed by covalently attaching an outer layer comprised of an ethylenically unsaturated prepolymer to an inner layer comprised of an ethylenically unsaturated polyelectrolyte and an enzyme. The final fused membrane composition acts a sensor membrane that provides a more stable and robust system. More specifically, the multilayered membrane formed comprises a restrictive domain that controls the flux of oxygen and glucose through the membrane to the working electrode without significant drift in sensor signal

Another aspect of the present invention is a method of making an analyte sensor, comprising the steps of disposing a sensing layer on a surface, treating the sensing layer with a coupling agent and attaching ethylenically unsaturated functional groups, and applying another layer over the sensing layer and curing the coated solution at a temperature range of between 4° C. to 80° C. The membrane being prepared from a composition reaction mixture of a polyelectrolyte prepolymer mixed with an enzyme and a crosslinker as a first layer that is functionalized with ethylenically unsaturated groups and chemically reacted with an outer layer comprised of an ethylenically unsaturated prepolymer.

Embodiments of the invention include a sensor having a plurality of layered elements including an analyte limiting membrane comprising a transition metal cured crosslinked silicone. Such polymeric membranes are particularly useful in the construction of electrochemical sensors for in vivo use, and embodiments of the invention include specific biosensor configurations that incorporate these polymeric membranes. The membrane embodiments of the invention allow for a combination of desirable properties including: permeability to molecules such as glucose over a range of temperatures, good mechanical properties of use as an outer polymeric membrane, and good processing properties for in situ preparation on a substrate. Consequently, glucose sensors that incorporate such polymeric membranes show an enhanced in vivo performance profile.

The ethylenically unsaturated silicone prepolymer may comprise about 40 to about 90 percent of the membrane. More specifically, the ethylenically unsaturated silicone prepolymer may comprise about 40, about 45, about 50, about 55, about 60, about 65, about 70, about 75, about 80, about 85 or about 90 percent of the membrane formed from the silicone composition reaction mixture.

The ethylenically unsaturated hydrophilic monomer may be comprised of hydroxy, alkoxy, epoxy, vinyl, and carboxylic acid end groups; and alkyl and ether main chain groups. More specifically, the monomer may be allyl alcohol, 2-allyloxyethanol, 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid, acrylic acid. Further, the ethylenically unsaturated monomer may comprise about 2 to about 30 percent of the membrane. More specifically, the ethylenically unsaturated monomer may comprise about 2, about 4, about 6, about 8, about 10, about 12, about 15, about 20, about 24, about 28 or about 30 percent of the membrane formed from the composition reaction mixture.

Another aspect of the present invention is an aqueous polymer composition comprising a polyelectrolyte prepolymer, wherein the percentage of the polyelectrolyte prepolymer is about 0.5 to about 20.0 and the molecular weight of the polyelectrolyte prepolymer is greater than 30,000 g/mol; an aziridine crosslinker wherein the percentage of the aziridine crosslinker is about 0.5 to about 20.0 and wherein the molecular weight of the aziridine is at least 100 g/mol and has at least two aziridine functional groups per molecule; and an enzyme wherein the percentage of the enzyme is about 0.5 to about 20.0, wherein the pH of the composition is between 3 and 8.

The percentage of the polyelectrolyte prepolymer in the composition may range from about 0.5 to about 20.0, about 1.0 to about 15, about 1.5 to about 10, or about 2.0 to about 7.0. More specifically, the percentage of the polyelectrolyte prepolymer may be 0.5, 1.0, 1.5, 2.0, 2.5, 3.0, 3.5, 4.0, 4.5, 5.0, 6.0, 7.0, 8.0, 9.0, 10.0, 12.0, 14.0, 16.0, 18.0 or 20.0. In addition, the molecular weight of the electrolyte prepolymer may range from about 30,000 to about 1,000,000, about 50,000 to about 800,000, about 100,000 to about 600,000, about 150,000 to about 500,000, about 200,000 to about 400,000 g/mol. More specifically, the molecular weight of the polyelectrolyte prepolymer is 30,000, 50,000, 70,000, 100,000, 150,000, 200,000, 250,000, 300,000, 350,000, 400,000, 450,000, 500,000, 600,000, 700,000, 800,000, 900,000 and 1,000,000 g/mol.

The percentage of aziridine in the composition may be from about 0.5 to about 20, about 1.0 to about 15, about 1.5 to about 10, about 2.0 to about 7.0. More specifically, the percentage of aziridine is 0.5, 1.0, 1.5, 2.0, 2.5, 3.0, 3.5, 4.0, 4.5, 5.0, 6.0, 7.0, 8.0, 9.0, 10.0, 12.0, 14.0, 16.0, 18.0 or 20.0. In addition, the aziridine molecule may have at least two aziridine functional groups. In one embodiment there are three functional groups on the aziridine.

The percentage of the enzyme in the composition may range from about 0.5 to about 20.0, about 1.0 to about 15, about 1.5 to about 10, or about 2.0 to about 7.0. More specifically, the percentage of the enzyme may be 0.5, 1.0, 1.5, 2.0, 2.5, 3.0, 3.5, 4.0, 4.5, 5.0, 6.0, 7.0, 8.0, 9.0, 10.0, 12.0, 14.0, 16.0, 18.0 or 20.0.

The pH of the composition may range from about 3 to about 8, about 4 to about 7, about 5 to about 6. More specifically, the pH may be 3, 4, 5, 6, 7 or 8.

In one embodiment, the aqueous polymer composition comprises a polyelectrolyte prepolymer, wherein the percentage of the polyelectrolyte prepolymer is about 5% and the molecular weight of the polyelectrolyte prepolymer is about 400,000 g/mol; an aziridine crosslinker wherein the percentage of the aziridine crosslinker is about 2% and wherein the molecular weight of the aziridine is at least 100 g/mol and has at least two aziridine functional groups per molecule; and an enzyme wherein the percentage of the enzyme is about 5%, wherein the pH of the composition is 5.

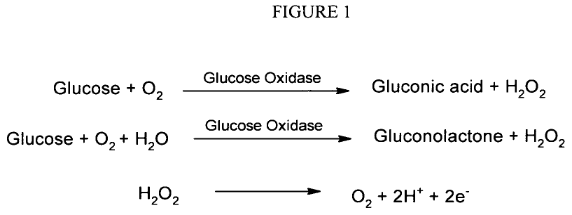

The continuous glucose monitoring system described herein is inserted underneath the skin with a small needle. The needle is removed and the sensor resides in the interstitial fluid and comes in direct contact with fluid containing glucose. The glucose permeates through the sensor membrane and reacts with glucose oxidase generating hydrogen peroxide that is then detected amperometrically (FIG. 1). Similar systems are described in In Vivo Glucose Sensing, Cunningham, D. D., Stenken, J. A., Eds; John Wiley & Sons, Hoboken, N.J., 2010.

The unexpected result is that when a hydrophilic enzyme polymer layer is formed with a methacrylate functional group creating a prepolymer, a second hydrophobic polymeric layer can be covalently attached to the enzyme layer through a polymerization reaction to provide a more stable and robust sensing system that has less drift than a standard multilayered membrane system that is not covalently bound to the other. More specifically, the ability to connect two different polymer layer phases (i.e., hydrophilic and hydrophobic) via a polymerization reaction was unexpected and had not previously been done.

### EXAMPLES

### Example 1

### Preparation of a Silicone Membrane-Coated Sensor

Preparation of silicone membrane dipping solution. Using two-part oleophilic reprographic silicone from Gelest, Inc. (Morrisville, Pa.), 7.36 g of part A (vinyl terminated polydimethylsiloxane) was mixed with 2.64 g of 2-hydroxyethyl methacrylate containing 2% diethyleneglycol dimethacrylate and 1.00 g of part B (methylhydrosiloxane-dimethylsiloxane copolymer, trimethylsiloxane terminated with vinyl, methyl modified silica). The mixture was speed mixed for 40 seconds.

Wire dipping with silicone solution. The dipping solution was transferred to a 1.5 mL plastic vial and placed under a dipping arm. The working electrode, a 0.003″ Pt wire, was covered with a layer of cross-linked glucose oxidase and dip coated with the silicone solution until a thickness of approximately 15μ was achieved. The coated wire was heated in an oven at 60° C. for 16 hours.

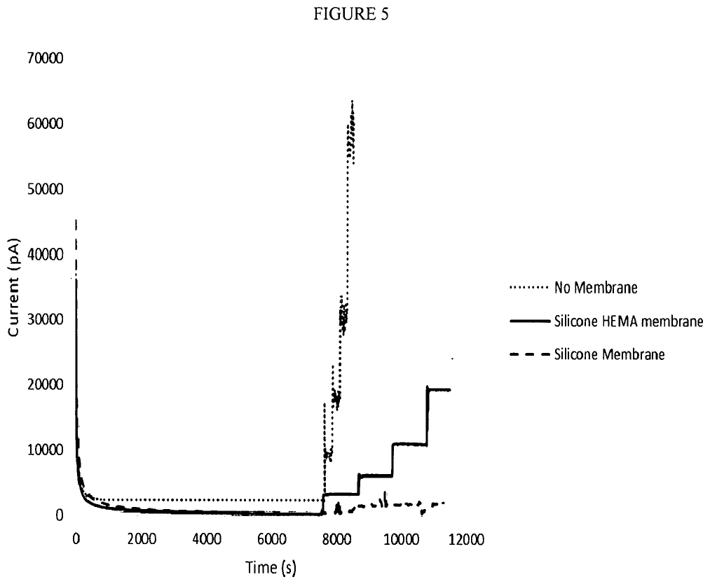

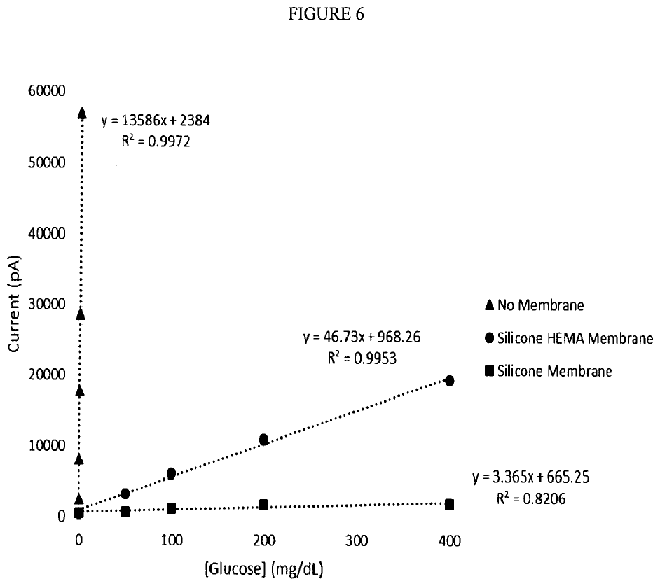

Testing of silicone membrane-coated wire. The silicone-based copolymer was evaluated as part of a two electrode electrochemical system. The counter and reference electrode was an iridium oxide coated wire. For comparison, 2 separate types of wires were prepared: one with crosslinked glucose oxidase but with no silicone membrane; and one with crosslinked glucose oxidase and silicone membrane containing no hydrophile. For each wire, the current was measured amperometrically and the electrochemical response was measured as a function of glucose concentration (FIG. 5). The concentration range of 0-400 mg/dL glucose was evaluated (FIG. 6).

| [glucose] (mg/dL) | No Membrane Current (pA) |
|---|---|
| 0 | 2462 |
| 0.5 | 8131 |
| 1 | 17737 |
| 2 | 28581 |
| 4 | 56906 |
| Sensitivity (pA/mg/dL) | 13586 |
| Baseline (pA) | 2384 |
| R² | 0.997 |

| | Silicone Membrane | Silicone-HEMA |
|---|---|---|
| [glucose] (mg/dL) | Current (pA) | Membrane Current (pA) |
| 0 | 466 | 328 |
| 50 | 710 | 3342 |
| 100 | 1181 | 6107 |
| 200 | 1684 | 10830 |
| 400 | 1809 | 19281 |
| Sensitivity (pA/mg/dL) | 3 | 52 |
| Baseline (pA) | 665 | 589 |
| R² | 0.821 | 0.996 |

The glucose response in vitro demonstrates the glucose limiting ability of the silicone membrane: without the membrane the glucose signal gave a sensitivity of 13586 pA/mg/dL with linearity up to 4 mg/dL glucose. With a silicone membrane containing no hydrophile the glucose signal gave a sensitivity of 3 pA/mg/dL with poor linearity (R²=0.8). The sensor wire built with the Silicone-HEMA membrane gave a sensitivity of 52 pA/mg/dL with linearity up to 400 mg/dL glucose.

### Example 2

### Preparation of a Chemically Fused Membrane Glucose Sensor

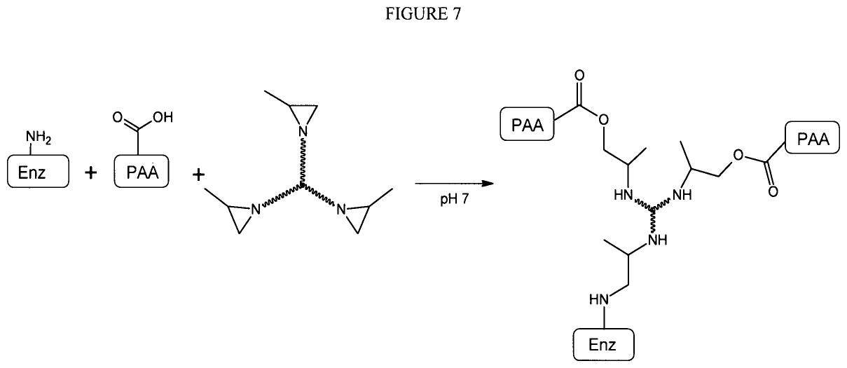

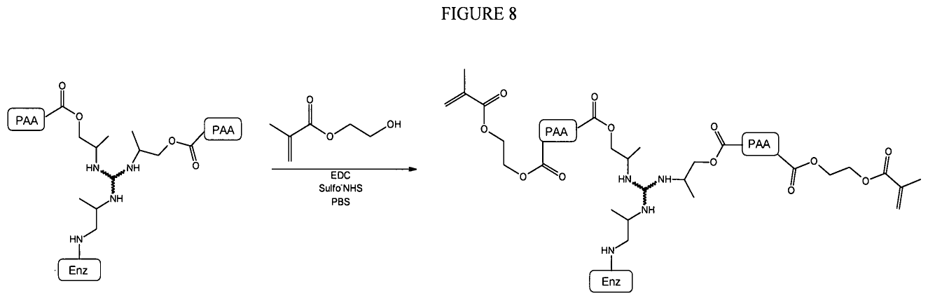

Preparation of an enzyme membrane dipping solution (FIGS. 7 and 8). Polyacrylic acid (PAA, MW 400,000, 10 g) was added to phosphate buffered saline (pH 7.0, 50 mM, 90 mL) and stirred for 16 hours at room temperature. In a separate container, 0.50 g glucose oxidase (GOX) was added to 5.00 g of pH 7.0 phosphate buffered saline (PBS). The solution was mixed with a speed mixer at 1400 rpm for 20 sec. Polyacrylic acid solution (5.00 g) was added to the GOX solution and mixed using a speed mixer set at 1400 rpm for 20 s. Trimethylolpropane tris(2-methyl-1-aziridine propionate) (0.1 g) was added into the GOX/PAA solution and mixed with a speed mixer set at 1400 rpm for 20 sec.

Dipping of enzyme solution on wire. Three 60 mm platinum wires were attached to a glass microscope slide such that 10 mm was exposed at the distal end of the wires. Using a dip coater the wires were dipped and dried until the wire OD+coating=85 μm thick (wire OD−Coating=2.5 μm). The slide with wires was placed in oven at 60° C. to cure for 2 hours.

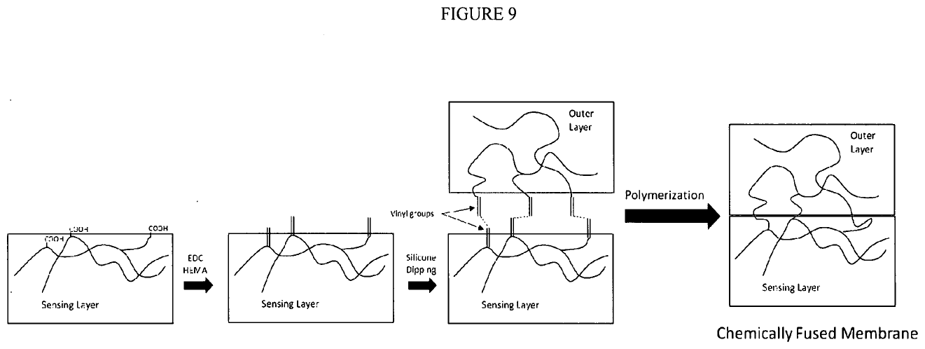

Preparation of 1-ethyl-3-(3-dimethylaminopropyl) carbodiimide (EDC) coupling solution (FIGS. 9A and B). 1-Ethyl-3-(3-dimethylaminopropyl) carbodiimide (156 mg) and phosphate buffered saline (pH 7.0, 50 mM, 10 mL) were added to a container and the mixture was vortexed. Sulfo-N-hydroxy succinimide (434 mg) was added along with 2-hydroxyethylmethacrylate (124 μL) and the mixture was stirred for 5 sec. with a vortex mixture.

Dipping of enzyme coated wire into EDC solution. A microscope slide with 3 enzyme coated wires with 4 mm of the distal end of the wires exposed were dipped into the EDC solution for 1.5 hours and then transferred to a PBS solution (pH 7.4, 50 mM, 10 mL). The wires were soaked in the PBS solution for 5 min. and then transferred to a 60° C. oven and dried for 20 min.

Preparation of Silicone Dipping Solution. Using two part oleophilic reprographic silicone from Gelest, Inc. (Morrisville, Pa.), 7.03 g of part A (vinyl terminated polydimethylsiloxane) was mixed with 2.97 g of 2-hydroxyethyl methacrylate containing 2% diethyleneglycol dimethacrylate and 1.00 g of part B (methylhydrosiloxane-dimethylsiloxane copolymer, trimethylsiloxane terminated with vinyl, methyl modified silica). The mixture was speed mixed for 40 seconds. Using two-part oleophilic reprographic silicone from Gelest, Inc. (Morrisville, Pa.), 7.03 g of part A (vinyl terminated polydimethylsiloxane) was mixed with 2.97 g of 2-hydroxyethyl methacrylate containing 2% diethyleneglycol dimethacrylate and 1.00 g of part B (methylhydrosiloxane-dimethylsiloxane copolymer, trimethylsiloxane terminated with vinyl, methyl modified silica). The mixture was speed mixed for 40 seconds.

Dipping of EDC-treated wires into silicone solution. The silicone dipping solution was transferred to a 40 mL plastic cup and placed under a dipping arm. The EDC-treated wires were dip-coated with the silicone solution until a thickness of approximately 15μ was achieved. The coated wire was heated in an oven at 60° C. for 16 hours.

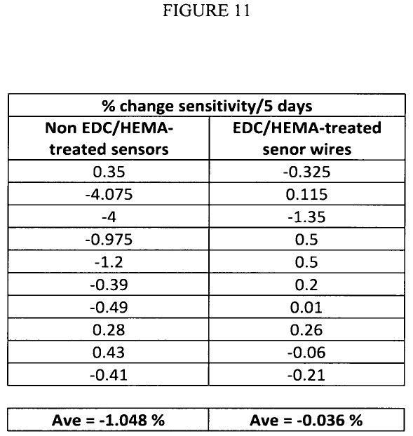

Testing of an EDC treated wire (FIG. 11). The EDC treated sensor wire that was coated with a silicone outer membrane was evaluated as part of a two electrode electrochemical system. The counter and reference electrode was an iridium oxide coated wire. For comparison, two sets of wire types were prepared: one that was not EDC/HEMA-treated; and one that was EDC/HEMA-treated. For each wire, the current was measured amperometrically and the electrochemical response was measured as a function of glucose concentration. The concentration range of 0-400 mg/dL glucose was evaluated.

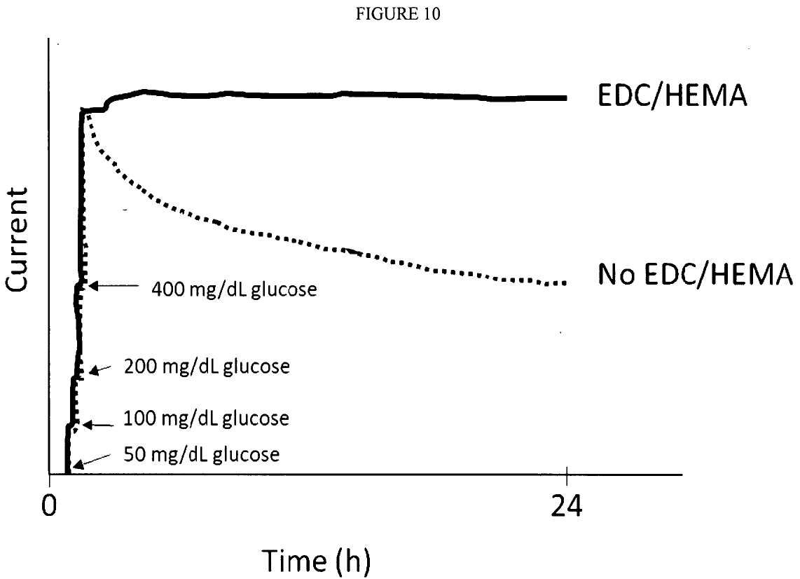

The glucose response in vitro demonstrates the signal stability ability of the EDC/HEMA treated membrane: without the membrane the average sensor sensitivity decreases by 1.3% over 5 days, whereas with EDC/HEMA treatment the average sensor sensitivity decreases by 0.036% (FIG. 10).

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

I claim:

1. An analyte sensor, comprising:
a working electrode; and
a membrane disposed over said electrode, said membrane formed from a silicone composition reaction mixture of:
an ethylenically unsaturated silicone prepolymer;
a hydride silicone prepolymer;
a non-silicone ethylenically unsaturated hydrophilic monomer;
a filler; and
a metal catalyst,
wherein the silicone composition formed from said silicone composition reaction mixture restricts diffusion of an analyte through said membrane, wherein said membrane comprises a restrictive domain and wherein said restrictive domain controls a flux of oxygen and glucose through said membrane and wherein said silicone composition formed has the structure, 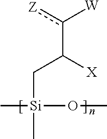
wherein
X is H or an alkyl;
Z is O, H₂;
W is OH, O-alkyl, O-alkylhydroxy, O-alkylalkoxy, O-methacrylate, O-acrylate, and
n is >1.

2. The analyte sensor according to claim 1, wherein said non-silicone ethylenically unsaturated hydrophilic monomer contains functional groups, wherein said functional groups are selected from the group consisting of hydroxy, ethoxy, methoxy, ethylene oxide, propylene oxide, methacrylate, acrylate, and/or carboxylic acids.

3. The analyte sensor according to claim 2, wherein said non-silicone ethylenically unsaturated hydrophilic monomer is 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid or acrylic acid.

4. The analyte sensor according to claim 2, wherein said non-silicone ethylenically unsaturated hydrophilic monomer is allyl alcohol or 2-allyloxyethanol.

5. The analyte sensor according to claim 1, wherein W is —OM, —O—(CH₂)ₘCH₃, —O(CH₂)₂—OH, —O—CH₂—(CH₂)₂O, —(O—CH₂CH₂)ₘ—O—C═O—C(CH₂)(CH₃), wherein m≥0 and M is Na, K or H.

6. An analyte sensor, comprising:
a working electrode; and
a membrane disposed over said electrode, said membrane formed from a silicone composition reaction mixture of:
an ethylenically unsaturated silicone prepolymer;
a hydride silicone prepolymer;
a non-silicone ethylenically unsaturated hydrophilic monomer;
a filler; and
a metal catalyst,
wherein the silicone composition formed from said silicone composition reaction mixture restricts diffusion of an analyte through said membrane, wherein said membrane comprises a restrictive domain and wherein said restrictive domain controls a flux of oxygen and glucose through said membrane and wherein said silicone composition formed has the structure, 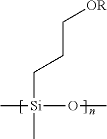
wherein
R is H or —(CH₂CH₂O)ₘ—CH₂CH₂OH;
m is ≥0; and
n is >1.

7. A method of making an analyte sensor, said method comprising steps of:
disposing a sensing layer on the surface of an electrode;
applying a membrane over the sensing layer by coating with a silicone solution comprised of:
an ethylenically unsaturated prepolymer;
a hydride silicone prepolymer;
a non-silicone ethylenically unsaturated hydrophilic monomer;
a filler; and
a metal catalyst and
curing said silicone solution coated on said surface of an electrode at a temperature of between 4° C. to 80° C.

8. An analyte sensor, comprising:
a working electrode; and
a multilayered membrane disposed over said electrode, said multilayered membrane having at least:
a sensing layer of an ethylenically unsaturated polyelectrolyte prepolymer disposed over said working electrode; and
a flux limiting layer, said flux limiting layer of an ethylenically unsaturated prepolymer and a hydride prepolymer disposed over said sensing layer, wherein said sensing layer and flux limiting layers are covalently attached to one another.

9. The analyte sensor according to claim 8, further comprising a reference electrode.

10. The analyte sensor according to claim 8, wherein the reference electrode contains iridium, iridium oxide, rhodium, or rhodium oxide.

11. The analyte sensor according to claim 8, wherein the sensing layer comprises an enzyme.

12. The analyte sensor according to claim 11, wherein the enzyme is an oxidase.

13. The analyte sensor according to claim 11, wherein the enzyme is glucose oxidase, lactate oxidase, glucose dehydrogenase, catalase, 3-hydroxybutyrate dehydrogenase, and/or β-hydroxybutyrate dehydrogenase.

14. The analyte sensor according to claim 8, wherein the ethylenically unsaturated polyelectrolyte prepolymer is a carboxylic acid.

15. The analyte sensor according to claim 14, wherein the carboxylic acid is a polyacrylic acid or a polyurethane.

16. The analyte sensor according to claim 8, wherein the sensing layer is formed through a crosslinking reaction.

17. The analyte sensor according to claim 16, wherein the crosslinker of said crosslinking reaction is an aziridine.

18. The analyte sensor according to claim 17, wherein said aziridine is trimethylolpropanetris(2-methyl-1-aziridinepropionate); pentaerythritoltris(3-(1-aziridinyl)propionate; or N,N′-(methylenedi-p-phenylene)bis(aziridine-1-carboxamide).

19. The analyte sensor according to claim 8, wherein said ethylenically unsaturated prepolymer of said flux limiting layer is an ethylenically unsaturated silicone prepolymer.

20. The analyte sensor according to claim 19, wherein the ethylenically unsaturated silicone prepolymer is vinyl functional polysiloxanes; ethylenoxide functional polysiloxanes, or tetrahydrofurfuryloxypropyl siloxanes.

21. The analyte sensor according to claim 8, wherein said flux limiting layer contains functional groups of hydroxy, ethoxy, methoxy, ethylene oxide, propylene oxide, methacrylate, acrylate, and/or carboxylic acids.

22. The analyte sensor according to claim 8, wherein said flux limiting layer comprises groups selected from 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid, acrylic acid, allyl alcohol, 2-allyloxyethanol, ethylenoxide terminated monovinylpolysiloxane and/or tetrahydrofurfuryloxypropyl terminated monovinylpolysiloxane.

23. The analyte sensor according to claim 8, further comprising a biocompatible layer disposed over said flux limiting layer of said multilayer membrane.

24. An aqueous polymer composition comprising:
a polyelectrolyte prepolymer, wherein the percentage of the polyelectrolyte prepolymer is about 0.5 to about 20.0 and the molecular weight of the polyelectrolyte prepolymer is greater than 30,000 g/mol;
an aziridine crosslinker wherein the percentage of the aziridine crosslinker is about 0.5 to about 20.0 and wherein the molecular weight of the aziridine is at least 100 g/mol and has at least two aziridine functional groups per molecule; and
an enzyme wherein the percentage of the enzyme is about 0.5 to about 20.0, wherein the pH of the composition is between 3 and 8.

25. An aqueous polymer composition comprising:
a polyelectrolyte prepolymer, wherein the percentage of the polyelectrolyte prepolymer is about 5 and the molecular weight of the polyelectrolyte prepolymer is about 400,000 g/mol;
an aziridine crosslinker wherein the percentage of the aziridine crosslinker is about 2 and wherein the molecular weight of the aziridine is at least 100 g/mol and has at least two aziridine functional groups per molecule; and
an enzyme wherein the percentage of the enzyme is about 5, wherein the pH of the composition is about 5.

26. The aqueous polymer composition according to claim 24, wherein said polyelectrolyte is polyacrylic acid or a polyurethane.

27. The aqueous polymer composition according to claim 24, wherein said enzyme is an oxidase.

28. The aqueous polymer composition according to claim 24, wherein said enzyme is a glucose oxidase, lactate oxidase, glucose dehydrogenase, catalase, 3-hydroxybutyrate dehydrogenase, or β-hydroxybutyrate dehydrogenase.

29. The aqueous polymer composition according to claim 24, wherein said aziridine crosslinker is trimethylolpropanetris(2-methyl-1-aziridinepropionate); pentaerythritoltris(3-(1-aziridinyl)propionate; or N,N′-(methylenedi-p-phenylene)bis(aziridine-1-carboxamide).

30. A method of making an analyte sensor, comprising the steps of:
disposing a first layer on a substrate; wherein said first layer is formed in a crosslinking reaction utilizing a crosslinker;
chemically modifying said first layer with ethylenically unsaturated groups; and
disposing a subsequent layer comprising an ethylenically unsaturated prepolymer; wherein said subsequent layer is formed in a chain-growth polymerization reaction.

31. The method according to claim 30, wherein said crosslinker is an aziridine.

32. The method according to claim 30, wherein said crosslinking reaction involves a carboxylic acid.

33. The method according to claim 32, wherein said carboxylic acid is a polyacrylic acid or a polyurethane.

34. The method according to claim 31, wherein the aziridine is trimethylolpropanetris(2-methyl-1-aziridinepropionate); pentaerythritoltris(3-(1-aziridinyl)propionate; or N,N′-(methylenedi-p-phenylene)bis(aziridine-1-carboxamide).

35. The method according to claim 30, wherein said chain-growth polymerization reaction is a platinum cured hydrosilyation reaction or a free radical reaction.

36. The method according to claim 35, wherein said free radical reaction is initiated by a photo-initiator or a thermal-initiator.
